(19) United States
(12) Patent Application Publication
(10) Pub. No.: US 2019/0000159 A1
(43) Pub. Date: Jan. 3, 2019

(54) THERMAL THERAPY GARMENT SYSTEM

(71) Applicant: Brandon Roberts, Centerville, OH (US)

(72) Inventor: Brandon Roberts, Centerville, OH (US)

(21) Appl. No.: 15/807,533

(22) Filed: Nov. 8, 2017

Related U.S. Application Data
(60) Provisional application No. 62/528,265, filed on Jul. 3, 2017.

(51) Int. Cl. A41D 13/005; A61F 7/10

(52) U.S. Cl. CPC A41D 13/0058; A61F 7/10; A61F 2007/023; A41D 13/0053; A41D 13/0051

(57) ABSTRACT

A thermal therapy garment system includes at least one garment assembly having a compression garment including an outer-surface, and an inner-surface having a plurality of pockets, and at least one thermal-insert. The plurality of pockets is located on the inner-surface of the compression garment and are strategically placed on the compression garment such that thermal therapy is enabled on at least one targeted area of a body of a user-wearer. The targeted area of the body of the user-wearer is at least one muscle, at least one joint, or a combination thereof. The compression garment may be worn underneath everyday clothing for all-day relief, enabling people to maintain a functional lifestyle.

### CROSS REFERENCE TO RELATED APPLICATION

The present application is related to and claims priority to U.S. Provisional Patent Application No. 62/528,265 filed Jul. 3, 2017, which is incorporated by reference herein in its entirety.

### BACKGROUND OF THE INVENTION

The following includes information that may be useful in understanding the present disclosure. It is not an admission that any of the information provided herein is prior art nor material to the presently described or claimed inventions, nor that any publication or document that is specifically or implicitly referenced is prior art.

### 1. Field of the Invention

The present invention relates generally to the field of therapy devices and more specifically relates to thermal therapeutic devices.

### 2. Description of Related Art

Many people use ice/heat packs on injuries. An ice pack is a portable plastic sac filled with water, or refrigerant gel or liquid. An ice pack is placed over an injured area and is intended to absorb heat of a closed traumatic or edematous injury. A heat pack is designed to warm up injuries to prevent swelling of injuries Both are commonly used to alleviate the pain of minor injuries, as well as decrease muscle soreness. Ice or heat pack therapy typically requires individuals to remain stationary or hold the packs on their body in an awkward position. Ice can begin to drip and leak over clothing and furniture, creating an extensive mess. People may start to get frustrated, dealing with applying ice/heat packs to the back, neck, legs, etc. Having to constantly apply ice or heat can begin impacting an individual's everyday life, leaving them unable to enjoy physical activities such as jogging, cycling, hiking, and more. A suitable solution is desired.

U.S. Pub. No. 2008/0125842 to Steve Petitt relates to a thermal, elastic, tight-fitting garment with pockets positioned for thermal therapy. The described thermal, elastic, tight-fitting garment with pockets positioned for thermal therapy includes a form-fitting elastic garment having a plurality of pockets at locations corresponding to a plurality of muscle groups and/or tendons. The pockets are adapted to receive a thermal medium and hold the thermal medium tightly against a muscle group and/or tendons of a wearer. A thermal therapy kit includes a thermally insulated container storing a thermal medium and such a form fitting garment.

### BRIEF SUMMARY OF THE INVENTION

In view of the foregoing disadvantages inherent in the known therapy means art, the present disclosure provides a novel thermal therapy garment system. The general purpose of the present disclosure, which will be described subsequently in greater detail, is to provide a series of form fitting garments including a partial turtleneck, t-shirt, compression pants, knee high socks, and ankle socks with built in compartments stitched into the garment for fitting ice and heat packs.

A thermal therapy garment system is disclosed herein. The thermal therapy garment system includes at least one garment assembly having a compression garment including an outer-surface, and an inner-surface having a plurality of pockets, and at least one thermal-insert configured to provide thermal therapy to a user-wearer.

The at least one garment assembly includes the compression garment and the at least one thermal-insert in functional combination, each of the at least one thermal-insert are configured to fit within at least one of the plurality of pockets. The plurality of pockets is located on the inner-surface of the compression garment. The plurality of pockets is strategically placed on the compression garment such that thermal therapy is enabled on at least one targeted area of a body of the user-wearer including muscles, muscle groups, and joints and the like. The compression garment may include a long-sleeved-shirt, a short-sleeved-shirt, pants, shorts, ankle-socks, high compression-socks, a vest, a shoulder and neck wrap, a sweater, or a jacket or combination thereof. The compression garment holds the at least one thermal-insert tightly against the targeted area of the body of the user. The targeted area of the body of the user-wearer is at least one muscle, at least one joint, or a combination thereof. The inner-surface is adjacent to the body of the user-wearer during an in-use condition.

A kit is also disclosed herein including compression garment(s) having a plurality of pockets, and at least one thermal-insert configured to be inserted within the plurality of pockets.

For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein. The features of the invention which are believed to be novel are particularly pointed out and distinctly claimed in the concluding portion of the specification. These and other features, aspects, and advantages of the present invention will become better understood with reference to the following drawings and detailed description.

### BRIEF DESCRIPTION OF THE DRAWINGS

The figures which accompany the written portion of this specification illustrate embodiments and methods of use for the present disclosure, a thermal therapy garment system, constructed and operative according to the teachings of the present disclosure.

FIG. 1A is a perspective view of the thermal therapy garment system during an ‘in-use’ condition, according to an embodiment of the disclosure.

FIG. 1B is a perspective view of the thermal therapy garment system, according to an embodiment of the present disclosure.

FIG. 2A is a perspective view of the thermal therapy garment system, according to an embodiment of the present disclosure.

FIG. 2B is a perspective view of the thermal therapy garment system, according to an embodiment of the present disclosure.

FIG. 3A is a perspective view of the thermal therapy garment system, according to an embodiment of the present disclosure.

FIG. 3B is a perspective view of the thermal therapy garment system, according to an embodiment of the present disclosure.

FIG. 4A is a perspective view of the thermal therapy garment system, according to an embodiment of the present disclosure.

FIG. 4B is a perspective view of the thermal therapy garment system, according to an embodiment of the present disclosure.

FIG. 5A is a perspective view of the thermal therapy garment system, according to an embodiment of the present disclosure.

FIG. 5B is a perspective view of the thermal therapy garment system, according to an embodiment of the present disclosure.

The various embodiments of the present invention will hereinafter be described in conjunction with the appended drawings, wherein like designations denote like elements.

### DETAILED DESCRIPTION

As discussed above, embodiments of the present disclosure relate to a therapy device and more particularly to a thermal therapy garment system as used to improve the thermal therapeutic devices.

Generally, the present invention provides users with compression garments equipped with small pockets designed to removably receive ice or heat packs for pain relief. It includes multiple compartments stitched (or otherwise fastened) into each garment, allowing wearers to target specific areas of the body for applying ice or heat packs. These compartments may be concealed on the inner-surface of the garment. This allows individuals to wear the garments and packs underneath everyday clothing for all-day relief, enabling people to maintain a functional lifestyle. This further utilizes five different garments including a neck/shoulder garment, a shirt, pants, high compression socks, and ankle socks, offering full-body relief for back pain, neck or shoulder pain, upper and lower leg pain, and foot pain. The pockets may be accessed on the inside of the garment. A user may remove the ice or heat packs once done and continue with normal activity wearing the garment of the present invention. The inside pockets are concealed and a user may wear the garment without ice or heat packs and the garment has the appearance of regular compression wear. Other garment-types may be available.

The present invention effectively eliminates the need to hold an ice or heat pack on the body or to use uncomfortable and hindering accessories in order to obtain hot or cold relief from aches and pains. Thermal therapy garment system is a set of modified compression garments integrated with multiple pockets or compartments designed to hold ice or heat packs. The garments can be comprised of a partial turtleneck, a compression shirt, a pair of pants, a pair of high compression socks, and a pair of low ankle socks. Each garment may include rectangular or square pockets sewn directly the garment; the ankle socks can feature a circular compartment capable of receiving a round ice pack. The partial turtleneck can be worn under a normal shirt and contains 4-8 thick elastic belt loops that will hold a customized, flexible ice or heat pack in place to treat an injured neck and/or trapezius muscle. The inside compartment of the turtleneck can contain the belt loops to hold the pack in place. A detachable hood option with a shirt and partial turtleneck. The hood may attach via VELCO or zipper fastener means and also contain ice/heat compartments that can help people cool down from workouts or mitigate migraines/headaches.

The compression shirt can be worn underneath a standard shirt and allows individuals to target core and back muscles for ice/heat cooling/heating relief. The pants can be worn underneath shorts, pants, or other garments and can be used to apply ice and heat to various portions of the upper leg and shin areas. The high socks can be used to apply therapy to an injured shin or calf area. Further, the ankle socks can apply ice or heat to an injured ankle area. Each garment can be constructed using polyester, cotton, spandex, elastin, and other suitable materials. The garments may be available in numerous sizes in order to accommodate all user-wearer needs and preferences. Exact size, measurement, construction, and design specifications may vary upon manufacturing.

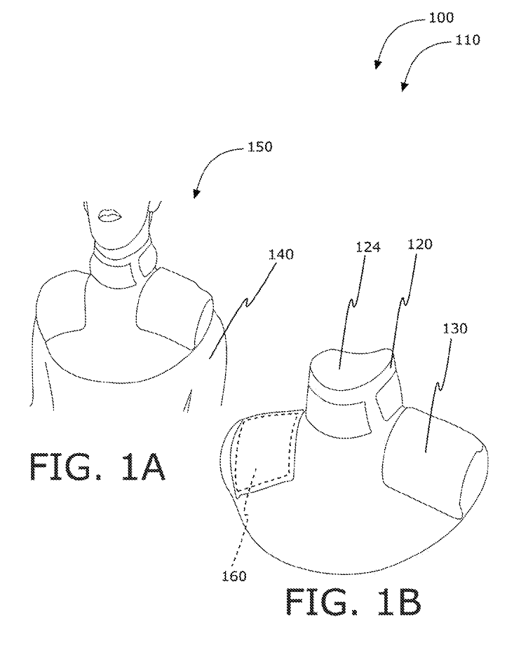

Referring now more specifically to the drawings by numerals of reference, there is shown in FIGS. 1A-5B, various views of a thermal therapy garment system 100. FIG. 1A shows a thermal therapy garment system 100 during an ‘in-use’ condition 150, according to an embodiment of the present disclosure. As illustrated, the thermal therapy garment system 100 may include at least one garment assembly 110 having a compression garment 120 including an outer-surface 128, and an inner-surface 124 a having a plurality of pockets 130, and at least one thermal-insert 160. The compression garment 120 may include a long-sleeved-shirt, a short-sleeved-shirt, pants, shorts, ankle-socks, high compression-socks, a vest, a shoulder and neck wrap, a sweater, and a jacket.

The at least one garment assembly 110 comprises the compression garment 120 and the at least one thermal-insert 160 in functional combination, each of the at least one thermal-insert 160 are configured to fit within at least one of the plurality of pockets 130. The plurality of pockets 130 are located on the inner-surface 124 of the compression garment 120. The plurality of pockets 130 are strategically placed on the compression garment 120 such that thermal therapy is enabled on at least one targeted area of a body of a user-wearer 140. The compression garment 120 holds the at least one thermal-insert 160 tightly against the targeted area of the body of the user-wearer 140. The inner-surface 124 is adjacent to the body of the user-wearer 140 during an in-use condition. The targeted area of the body of the user-wearer 140 is at least one muscle, at least one joint, or a combination thereof.

In certain embodiments the plurality of pockets 130 may be removeably coupled to the compression garment 120 via at least one fastener. The fastener is selected from the group consisting of hook-and-loop-fasteners, snap-fasteners, and zipper-fasteners. The compression garment 120 comprises a shoulder and neck wrap having the plurality of pockets 130 disposed at the targeted area of the body of the user-wearer 140 including a neck-area and trapezius-muscles. The compression garment 120 comprises a detachable hood having at least one of the plurality of pockets 130 configured to receive the thermal-insert 160. The plurality of pockets 130 comprise belt loops configured to secure the at least one thermal-insert 160.

The at least one thermal-insert 160 comprises a heat-pack-insert or an ice-pack-insert. The thermal-inserts 160 may be chosen based on user-preference and therapy needs. The thermal-inserts 160 may be used selectively and in combination. The plurality of pockets 130 compressingly conceal and hold adjacent the at least one thermal-insert 160 tightly against the targeted area of the body of the user-wearer 140. The compression garment 120 is configured to be worn underneath street clothing so that individuals may maintain their lifestyle while using the device for pain relief.

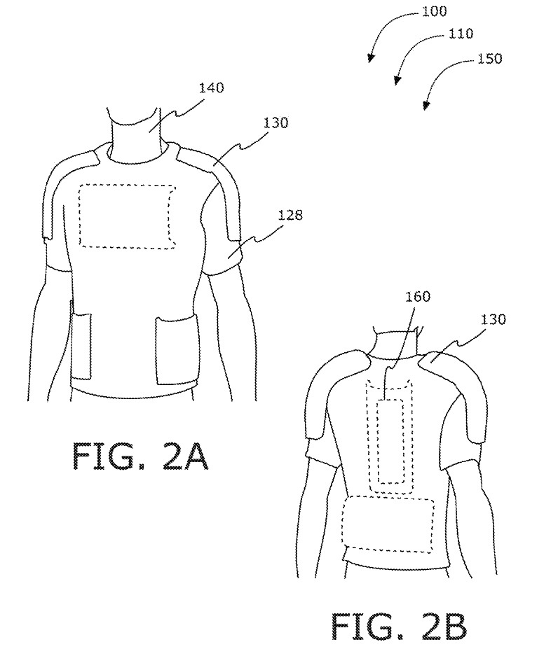

FIGS. 2A-2B show views of the thermal therapy garment system 100, according to an embodiment of the present disclosure. As above, the thermal therapy garment system 100 may include at least one garment assembly 110 having the compression garment 120 including the outer-surface 128, and the inner-surface 124 having the plurality of pockets 130. The compression garment 120 may include a short-sleeved-shirt as shown in FIGS. 2A-2B having the plurality of pockets 130 disposed at the targeted area of the body of the user-wearer 140 including shoulders, chest, upper-back, lower-back, and sides. The compression garment 120 may further comprise a long-sleeved-shirt having the plurality of pockets 130 positioned at a user-wearer's 140 biceps, triceps, elbows, anterior forearms, posterior forearms, and wrists.

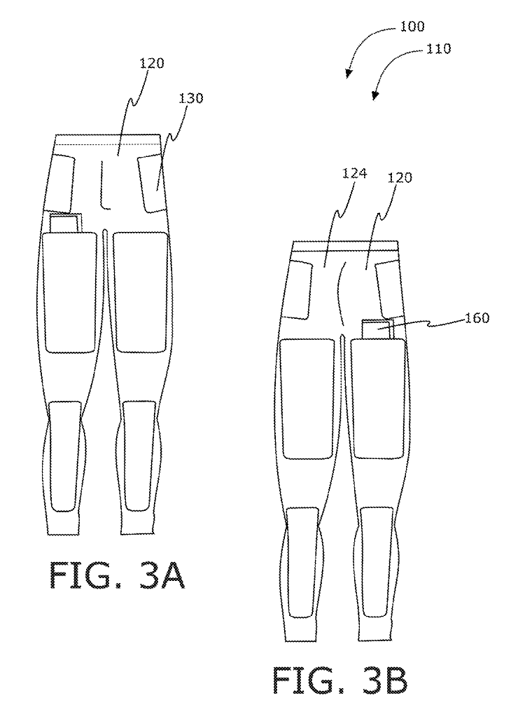

FIGS. 3A-3B show perspective views of the thermal therapy garment system 100, according to an embodiment of the present disclosure. As above, the thermal therapy garment system 100 may include at least one garment assembly 110 having the compression garment 120 including the outer-surface 128, and the inner-surface 124 having the plurality of pockets 130, and at least one thermal-insert 160. The compression garment 120 may include pants having the plurality of pockets 130 positioned at the targeted area of the body of the user-wearer 140 including a hip, a quadricep, a hamstring, a shin, and a calf. A heat-pack-insert or an ice-pack-insert may be inserted selectively within the plurality of pockets 130 to provide pain relief to desired targeted area of the body.

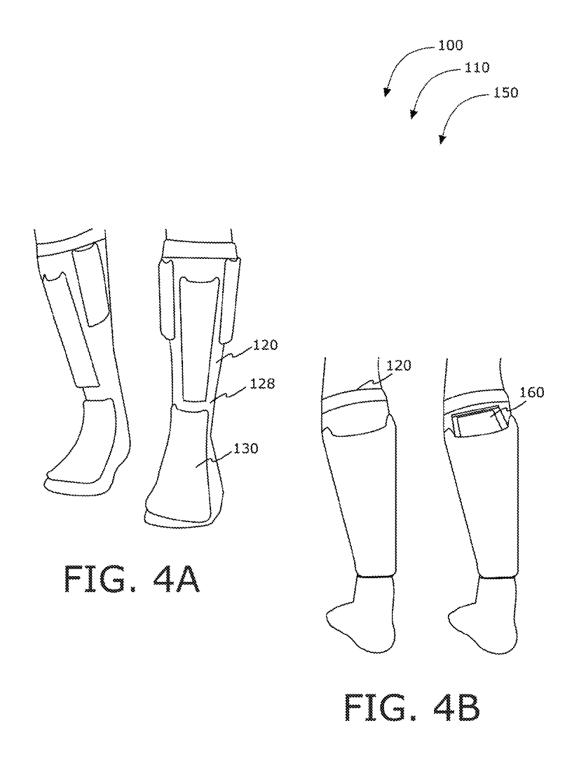

FIGS. 4A-4B show perspective views of the thermal therapy garment system 100, according to an embodiment of the present disclosure. As above, the thermal therapy garment system 100 may include at least one garment assembly 110 having the compression garment 120 having the plurality of pockets 130 configured to receive at least one thermal-insert 160. The compression garment 120 may include high compression-socks having the plurality of pockets 130 disposed at the targeted area of the body of the user-wearer 140 including a shin, a calf, an ankle, and a foot. The plurality of pockets 130 may vary in size to accommodate larger muscles and joints.

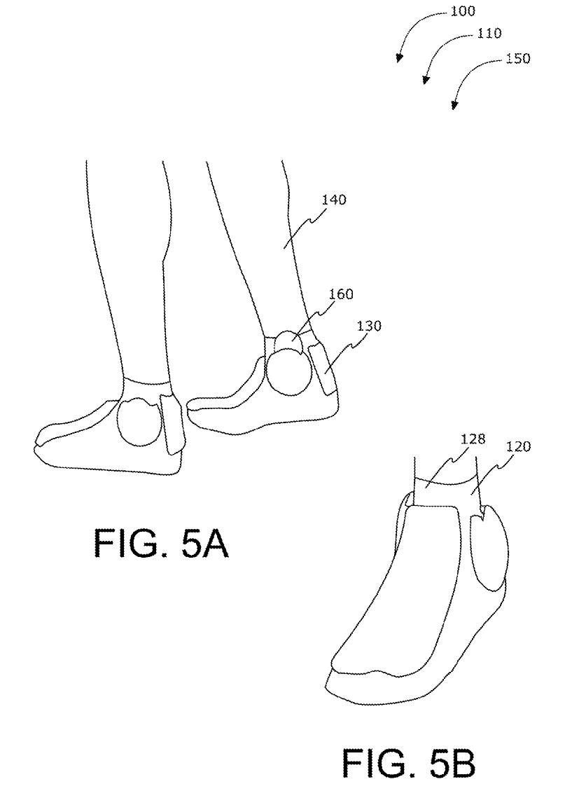

FIGS. 5A-5B show perspective views of the thermal therapy garment system 100, according to an embodiment of the present disclosure. As above, the thermal therapy garment system 100 may include the compression garment 120 having the plurality of pockets 130, and at least one thermal-insert 160. The compression garment 120 as shown in FIGS. 5A-5B, may include ankle-socks having the plurality of pockets 130 disposed at the targeted area of the body of the user-wearer 140 including an ankle and a foot area. The plurality of pockets 130 may include a circular profile and may be disposed on the ankle on opposing sides. Square or rectangular plurality of pockets 130 may be positioned on a front and back side of the foot.

A kit is also disclosed herein including compression garment(s) 120 having a plurality of pockets 130, and at least one thermal-insert 160 configured to be inserted within the plurality of pockets 130. Compression garment(s) 120 may comprise various combinations of garments disclosed and thermal-insert(s) 160.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

What is claimed is new and desired to be protected by Letters Patent is set forth in the appended claims:

1. A thermal therapy garment system comprising:
at least one garment assembly having;
a compression garment including;
an outer-surface; and
an inner-surface having;
a plurality of pockets; and
at least one thermal-insert;
wherein said at least one garment assembly comprises said compression garment and said at least one thermal-insert in functional combination, each of said at least one thermal-insert configured to fit within at least one of said plurality of pockets;
wherein said plurality of pockets are located on said inner-surface of said compression garment, said plurality of pockets strategically placed on said compression garment such that thermal therapy is enabled on at least one targeted area of a body of a user-wearer;
wherein said compression garment holds said at least one thermal-insert tightly against said targeted area of said body of said user-wearer, said at least one thermal-insert is concealed within the garment; and
wherein said inner-surface is adjacent to said body of said user-wearer during an in-use condition.

2. The thermal therapy garment system of claim 1, wherein said targeted area of said body of said user-wearer is at least one muscle.

3. The thermal therapy garment system of claim 2, wherein said targeted area of said body of said user-wearer is at least one joint.

4. The thermal therapy garment system of claim 1, wherein said compression garment is selected from the group consisting of a long-sleeved-shirt, a short-sleeved-shirt, pants, shorts, ankle-socks, high compression-socks, a vest, a shoulder and neck wrap, a sweater, and a jacket.

5. The thermal therapy garment system of claim 1, wherein said plurality of pockets are removeably coupled to said compression garment via at least one fastener.

6. The thermal therapy garment system of claim 5, wherein said fastener is selected from the group consisting of hook-and-loop-fasteners, snap-fasteners, and zipper-fasteners.

7. The thermal therapy garment system of claim 1, wherein said compression garment comprises shoulder and neck wrap having said plurality of pockets disposed at said targeted area of said body of said user-wearer including a neck-area and trapezius-muscles.

8. The thermal therapy garment system of claim 7, wherein said compression garment comprises a detachable hood having at least one of said plurality of pockets configured to receive said thermal-insert.

9. The thermal therapy garment system of claim 8, wherein said plurality of pockets comprise belt loops configured to secure said at least one thermal-insert.

10. The thermal therapy garment system of claim 1, wherein said compression garment comprises a long-sleeved-shirt having said plurality of pockets disposed at said targeted area of said body of said user-wearer including biceps, triceps, elbows, anterior forearms, posterior forearms, and wrists.

11. The thermal therapy garment system of claim 1, wherein said compression garment comprises pants having said plurality of pockets disposed at said targeted area of said body of said user-wearer including a hip, a quadricep, a hamstring, a shin, and a calf.

12. The thermal therapy garment system of claim 1, wherein said compression garment comprises ankle-socks having said plurality of pockets disposed at said targeted area of said body of said user-wearer including an ankle and a foot area.

13. The thermal therapy garment system of claim 1, wherein said compression garment comprises high compression-socks having said plurality of pockets disposed at said targeted area of said body of said user-wearer including a shin, a calf, an ankle, and a foot.

14. The thermal therapy garment system of claim 1, wherein said compression garment comprises a short-sleeved-shirt having said plurality of pockets disposed at said targeted area of said body of said user-wearer including shoulders, chest, and back.

15. The thermal therapy garment system of claim 1, wherein said at least one thermal-insert comprises a heat-pack-insert.

16. The thermal therapy garment system of claim 1, wherein said at least one thermal-insert comprises an ice-pack-insert

17. The thermal therapy garment system of claim 1, wherein said plurality of pockets compressingly conceal and hold adjacent said at least one thermal-insert tightly against said targeted area of said body of said user-wearer.

18. The thermal therapy garment system of claim 1, wherein said compression garment is configured to be worn underneath street clothing.

19. A thermal therapy garment system, said thermal therapy garment system comprising:
at least one garment assembly having;
a compression garment including;
an outer-surface; and
an inner-surface having;
a plurality of pockets; and
at least one thermal-insert;
wherein said at least one garment assembly comprises said compression garment and said at least one thermal-insert in functional combination, each of said at least one thermal-insert configured to fit within at least one of said plurality of pockets;
wherein said plurality of pockets are located on said inner-surface of said compression garment, said plurality of pockets strategically placed on said compression garment such that thermal therapy is enabled on at least one targeted area of a body of a user-wearer;
wherein said compression garment is selected from the group consisting of a long-sleeved-shirt, a short-sleeved-shirt, pants, shorts, ankle-socks, high compression-socks, a vest, a shoulder and neck wrap, a sweater, and a jacket;
wherein said compression garment comprises a shoulder and neck wrap having said plurality of pockets disposed at said targeted area of said body of said user-wearer including a neck-area and trapezius-muscles;
wherein said plurality of pockets comprise belt loops configured to secure said at least one thermal-insert;
wherein said compression garment comprises a detachable hood having at least one of said plurality of pockets configured to receive said thermal-insert;
wherein said plurality of pockets are removeably coupled to said compression garment via at least one fastener;
wherein said fastener is selected from the group consisting of hook-and-loop-fasteners, snap-fasteners, and zipper-fasteners;
wherein said compression garment comprises a long-sleeved-shirt having said plurality of pockets disposed at said targeted area of said body of said user-wearer including biceps, triceps, elbows, anterior forearms, posterior forearms, and wrists;
wherein said compression garment comprises pants having said plurality of pockets disposed at said targeted area of said body of said user-wearer including a hip, a quadricep, a hamstring, a shin, and a calf;
wherein said compression garment comprises ankle-socks having said plurality of pockets disposed at said targeted area of said body of said user-wearer including an ankle and a foot area;
wherein said compression garment comprises high compression-socks having said plurality of pockets disposed at said targeted area of said body of said user-wearer including a shin, a calf, an ankle, and a foot;
wherein said compression garment comprises a short-sleeved-shirt having said plurality of pockets disposed at said targeted area of said body of said user-wearer including shoulders, chest, and back;
wherein said compression garment holds said at least one thermal-insert tightly against said targeted area of said body of said user-wearer;
wherein said targeted area of said body of said user-wearer is at least one muscle and alternatively at least one joint;
wherein said plurality of pockets compressingly conceal and hold adjacent said at least one thermal-insert tightly against said targeted area of said body of said user-wearer;
wherein said inner-surface is adjacent to said body of said user-wearer during an in-use condition; and
wherein said compression garment is configured to be worn underneath street clothing.

20. A thermal therapy garment system comprising a kit including:
at least one compression garment having a plurality of pockets;
at least one thermal-insert; and
a set of user instructions.
